(19) United States
(12) Patent Application Publication
(10) Pub. No.: US 2013/0196907 A1
(43) Pub. Date: Aug. 1, 2013

(54) Promoting Erythropoietin, Erythrocyte, and Hematopoietic Stem Cell Production By Activating HIF In OsteoBlasts

(71) Applicant: The Board of Trustees of the Leland Standford Junior University, Palo Alto, CA (US)

(72) Inventors: Erinn Bruno Rankin, Waltham, MA (US); Amato J. Giaccia, Stanford, CA (US)

(73) Assignee: The Board of Trustees of the Leland Stanford Junior University, Palo Alto, CA (US)

(21) Appl. No.: 13/641,417

(22) PCT Filed: Apr. 15, 2011
(86) PCT No.: PCT/US2011/032759

§ 371 Date: Mar. 21, 2013

Related U.S. Application Data
(60) Provisional application No. 61/325,180, filed on Apr. 16, 2010.

(51) Int. Cl. A61K 38/02; A61K 31/713; A61K 31/7088

(52) U.S. Cl. CPC A61K 38/02; A61K 31/7088; A61K 31/713
USPC 514/7.7; 435/375; 514/44 R; 435/377; 514/44 A

(57) ABSTRACT

Methods are provided for promoting the production of erythropoietin and the production of erythrocytes and hematopoietic stem and progenitor cells. These methods find us in the treatment of subjects with conditions in which erythrocyte cell numbers are reduced, for example, anemia, chronic kidney disease, and following chemotherapy treatment.

### FIELD OF THE INVENTION

This invention pertains to the use of agents to promote the production of erythropoietin, erythrocytes, and hematopoietic stem cells.

### SUMMARY OF THE INVENTION

Methods are provided for promoting the production of erythropoietin and the production of erythrocytes and hematopoietic stem and progenitor cells. These methods find us in the treatment of subjects with conditions in which erythrocyte cell numbers are reduced, for example, anemia, chronic kidney disease, and following chemotherapy treatment.

In one aspect of the invention, methods are provided for promoting the production of erythropoietin (EPO) comprising the steps of contacting an osteoblast with a hypoxia inducible factor (HIF) activating agent under conditions sufficient to promote the survival of said osteoblast, wherein EPO is produced. In some embodiments, the HIF activating agent promotes the transcriptional activity of a hypoxia inducible factor (HIF). In some embodiments, the transcriptional activity of a HIF is promoted by stabilizing the HIF. In some embodiments, the HIF being acted upon by the HIF activating agent is hypoxia inducible factor 1 alpha (HIF1A) (GenBank Accession Nos. NM_{—}001530 and NM_{—}181054), hypoxia inducible factor 2 alpha (HIF2A/EPAS1) (GenBank Accession No. NM_{—}001430), and/or hypoxia inducible factor 3 alpha (HIF3A) (GenBank Accession Nos. NM_{—}022462, NM_{—}152794 and NM_{—}152795). In some embodiments, the contacting step occurs in vitro. In some embodiments, the contacting step occurs in vivo. In some such embodiments, the HIF activating agent is administered directly to the bone marrow.

In one aspect of the invention, methods are provided for promoting the production of cells of the hematopoietic lineage from a population of hematopoietic progenitor cells, comprising the steps of contacting a population of osteoblasts with a HIF activating agent, and culturing a population of hematopoietic progenitor cells in the presence of said osteoblast population under conditions sufficient to promote the survival and differentiation of said hematopoietic progenitor cell population, wherein cells of the hematopoietic lineage are produced. In some embodiments, both the contacting step and the culturing step occur in vitro, and the contacting step occurs concurrently with the contacting step. In some embodiments, both the contacting step and the culturing step occur in vitro, and the contacting step occurs prior to said culturing step. In some embodiments, the contacting step occurs ex vivo and the culturing step occurs in vivo, and the contacting step occurs prior to the culturing step. In some embodiments, both the contacting step and the culturing step occur in vivo, and the contacting step occurs concurrently with the contacting step. In some embodiments in which the contacting step occurs in vivo, the HIF activating agent is administered directly to the bone marrow. In some embodiments, the cells of the hematopoietic lineage that are produced comprise erythrocytes. In some such embodiments, the number of erythrocytes that is produced is increased relative to the number of erythrocytes that would be produced in the absence of said HIF activating agent. In some embodiments, said cells of the hematopoietic lineage that are produced comprise hematopoietic stem and/or progenitor cells. In some such embodiments, number of hematopoietic stem and/or progenitor cells that is produced is increased relative to the number of hematopoietic stem and/or progenitor cells that would be produced in the absence of said HIF activating agent. In some embodiments, HIF activating agent promotes the stability of HIF. In some embodiments, HIF activating agent promotes the transcriptional activity of HIF.

In one aspect of the invention, methods are provided for increasing the number of erythrocytes in a subject in need thereof, comprising the steps of contacting osteoblasts with a HIF activating agent, and growing said osteoblasts in the presence of hematopoietic progenitor cells in the subject, wherein the number of erythrocytes is increased relative to the number of erythrocytes in the absence of the HIF activating agent. In some embodiments, the contacting step occurs ex vivo prior to the culturing step. In some embodiments, the contacting step occur in vivo concurrently with the culturing step. In some embodiments, the HIF activating agent is administered directly to the bone marrow. In some embodiments, erythrocyte production is promoted. In some embodiments, erythrocytes survival is prolonged. In some embodiments, the percent of circulating erythrocytes is increased relative to the total number of circulating hematopoietic cells or a subpopulation thereof. In some embodiments, the contacting step results in the production of EPO. In some embodiments, the subject has received or will receive chemotherapy or radiation therapy for cancer, has anemia, or has chronic kidney disease.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Hypoxia Signaling Pathway. (a) In the presence of VHL, HIFα is targeted for ubiquitin dependent degradation. (b) In the absence of VHL, HIFα is stabilized and able to travel to the nucleus, where it activates the transcription of genes.

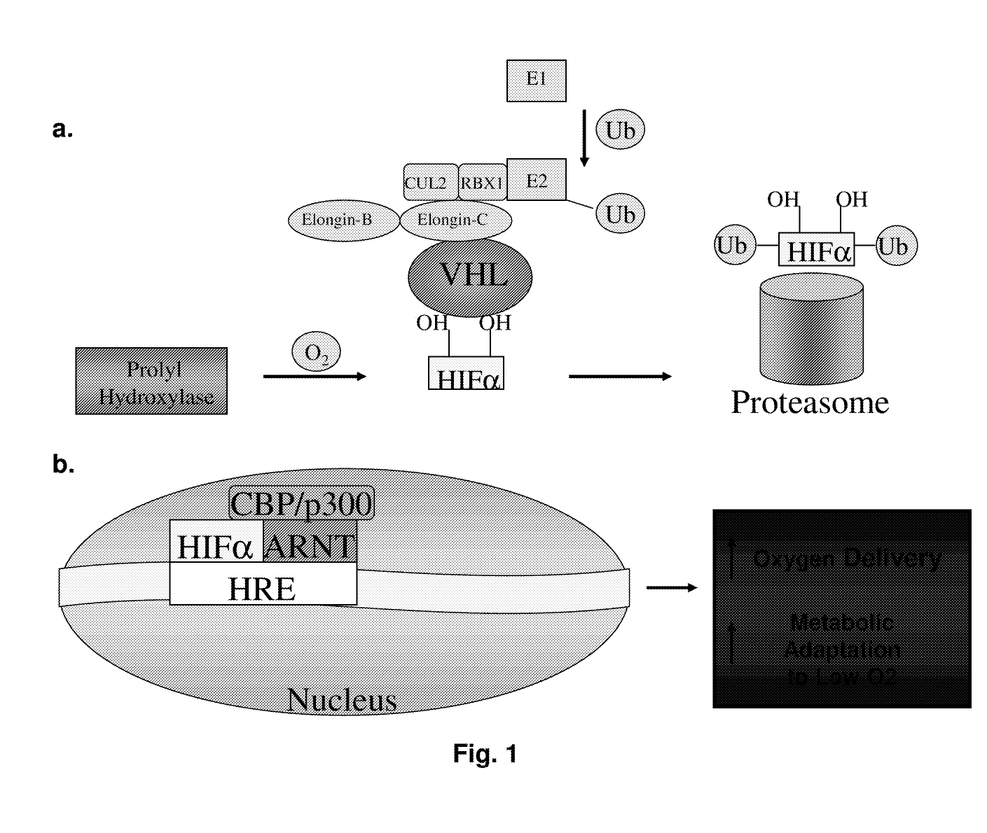

FIG. 2. VHL deletion in cells of the osteoblast lineage by breeding an Osterix promoter-driven cre transgenic mouse (Rodda S J et al., Development 2006) with a mouse homozygote for a floxed VHL allele (VHLfl/fl) (“OSX-VHL mutant mouse”) results in increased trabecular bone. After euthanasia, the long bones (femurs and tibias) was excised from the body of the mice and fixed in a 10% paraformaldehyde solution for 6 hours. The long bones were then decalcified in a 20% sodium EDTA (pH 7.4) for at least one week. The bone specimens were dehydrated by incubation in a series of ethanol solutions. Dehydrated bone specimens were be embedded in paraffin under vacuum and hen sectioned into 5-μm sections with an ultramicrotome. Sections were stained with H&E as well as for specific cell surface lineage markers.

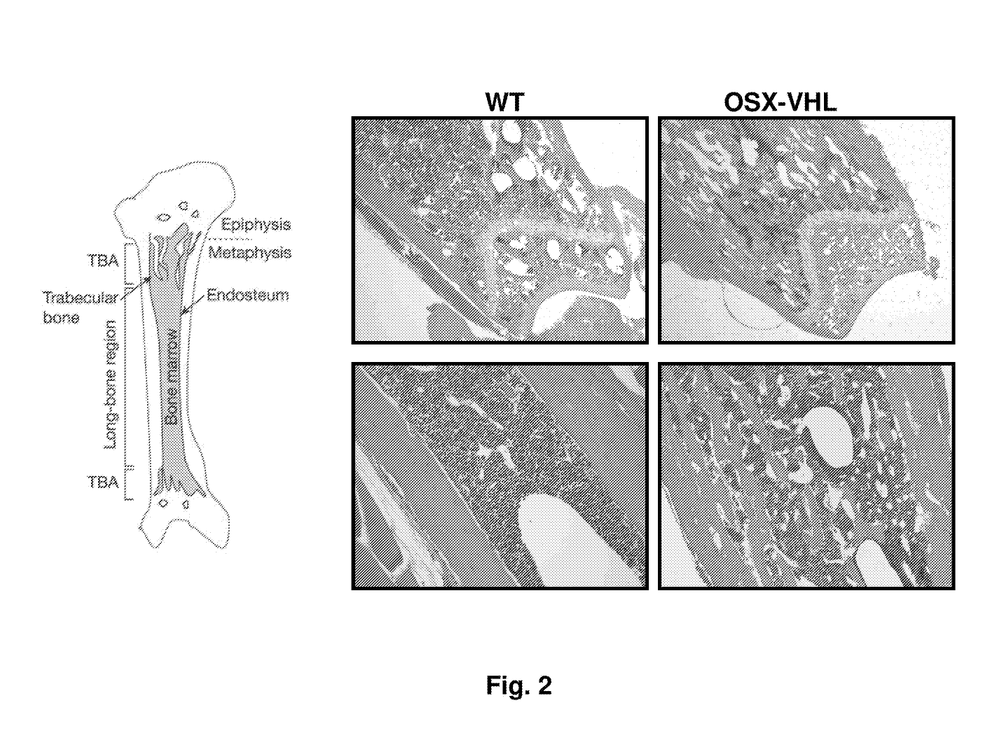

FIG. 3. VHL deletion in osteoprogenitors results in increased hematopoietic stem cells (Lin⁻Sca⁺Kit^{hi} (LSK)) in the bone marrow.

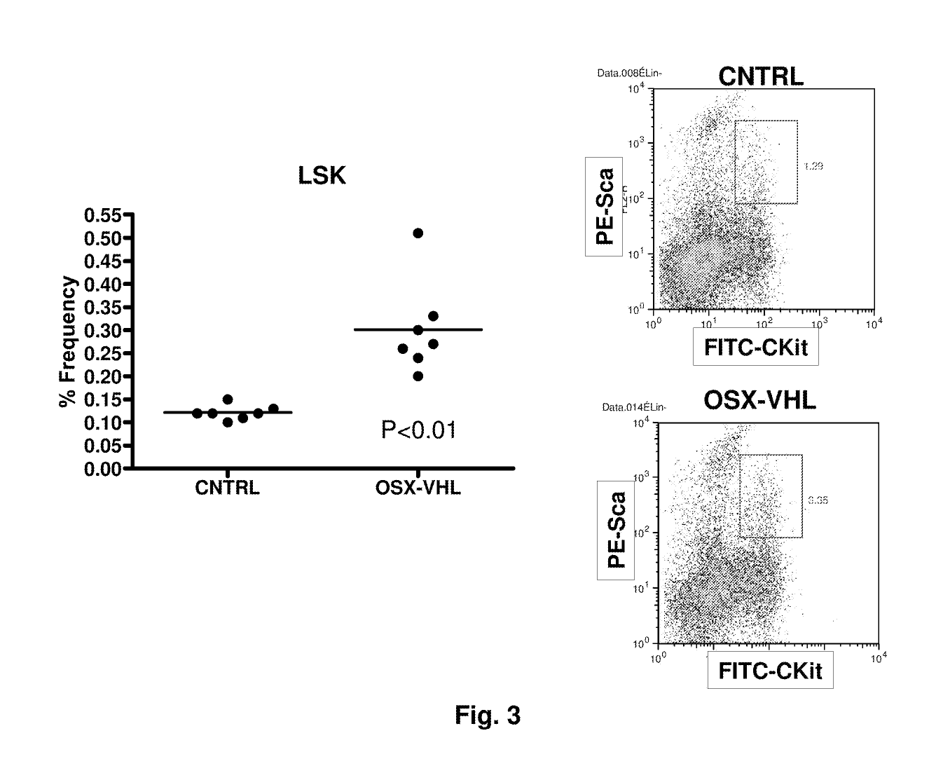

FIG. 4. OSX-VHL mutant mice develop polycythemia. For blood analysis, hematocrit values (HCT), red blood cells (RBC), and hemoglobin (HGB) concentrations were quantified using a Hemavet 1500 CBC analyzer (CDC Technologies). Hematocrit values were also determined by centrifuging blood in microcapillary tubes.

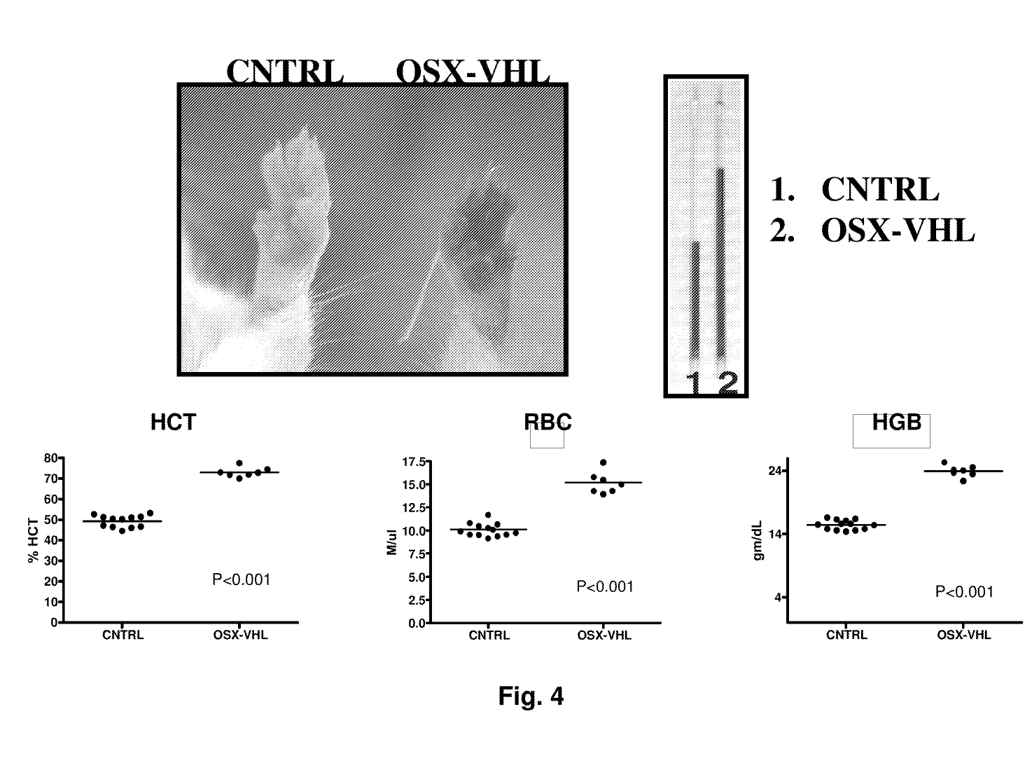

FIG. 5. Polycythemia in OSX-VHL mutant mice is associated with no change in numbers of neutrophils, platelets, or monocytes, but a decrease in the numbers of lymphocytes.

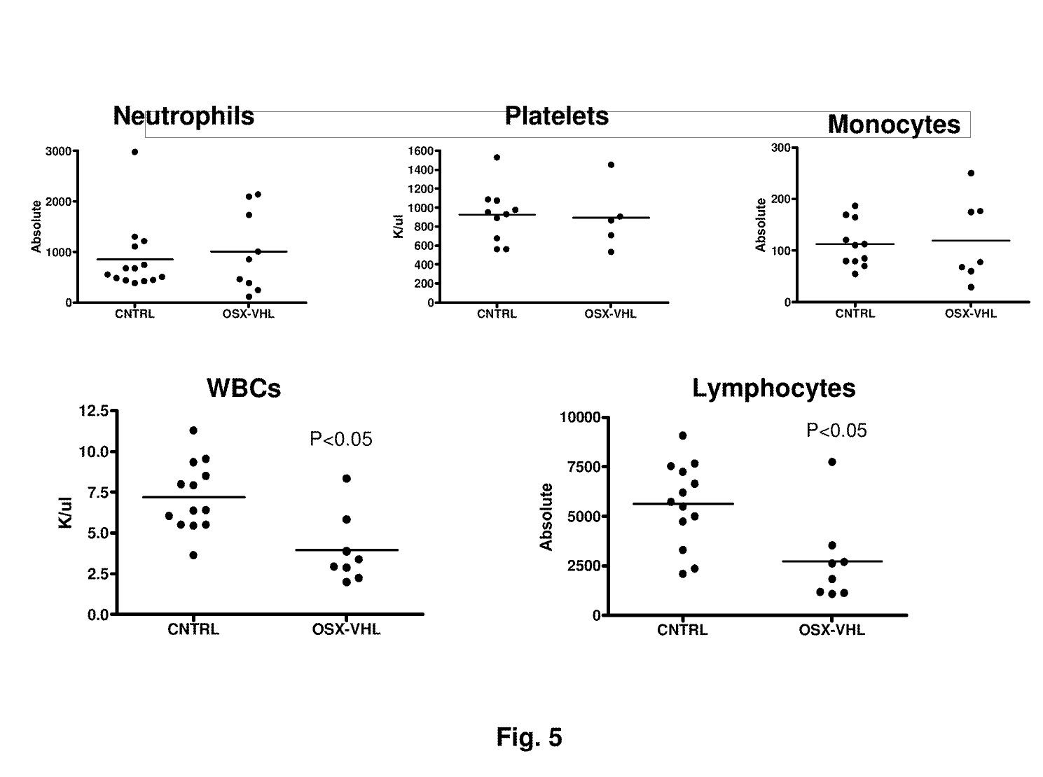

FIG. 6. Erythroid cells (Ter118+) are increased in OSX-VHL mutant bone marrow.

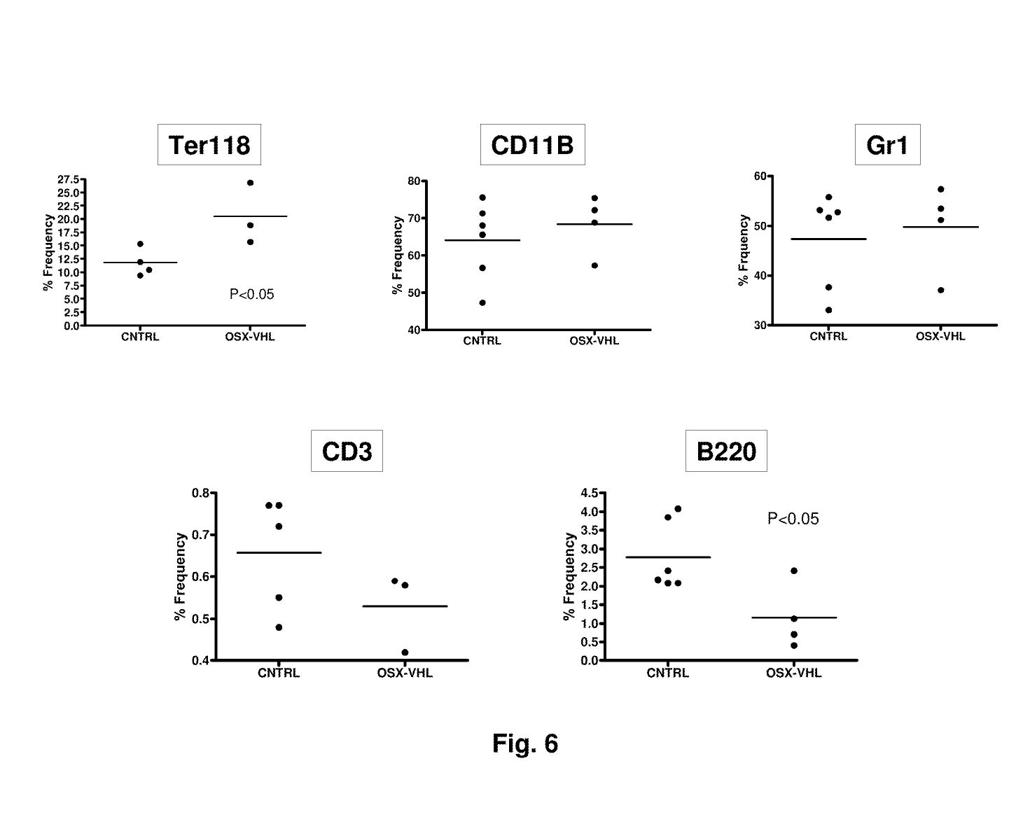

FIG. 7. Schematic of the animal crosses performed to determine if HIF signaling is required for increased HSC expansion and polycythemia in OSX-VHL mutant mice.

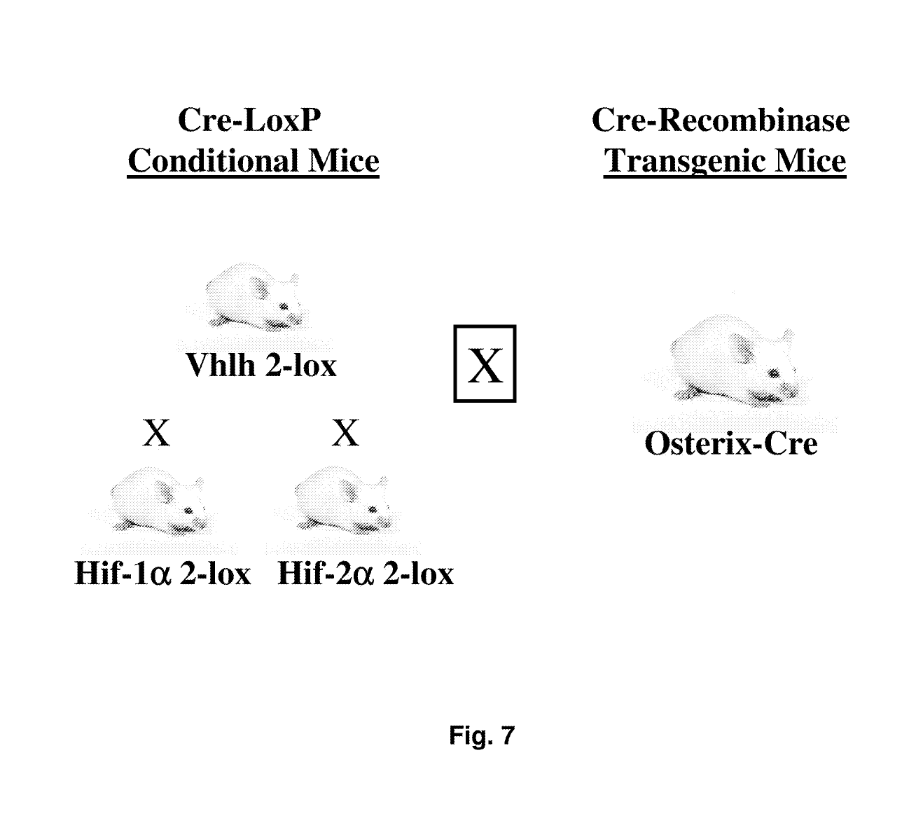

FIG. 8. HIF signaling is required for the induction of polycythemia in OSX-VHL mutant mice.

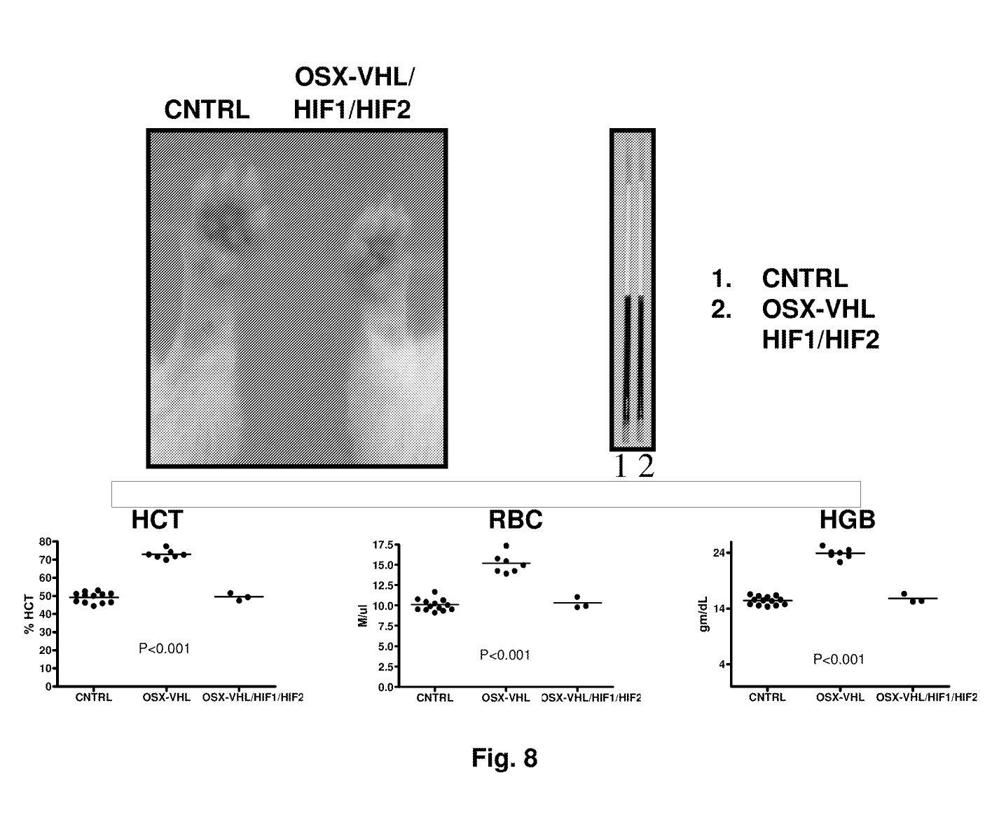

FIG. 9. HIF signaling is required for the erythroid expansion observed in OSX-VHL mutant bone marrow.

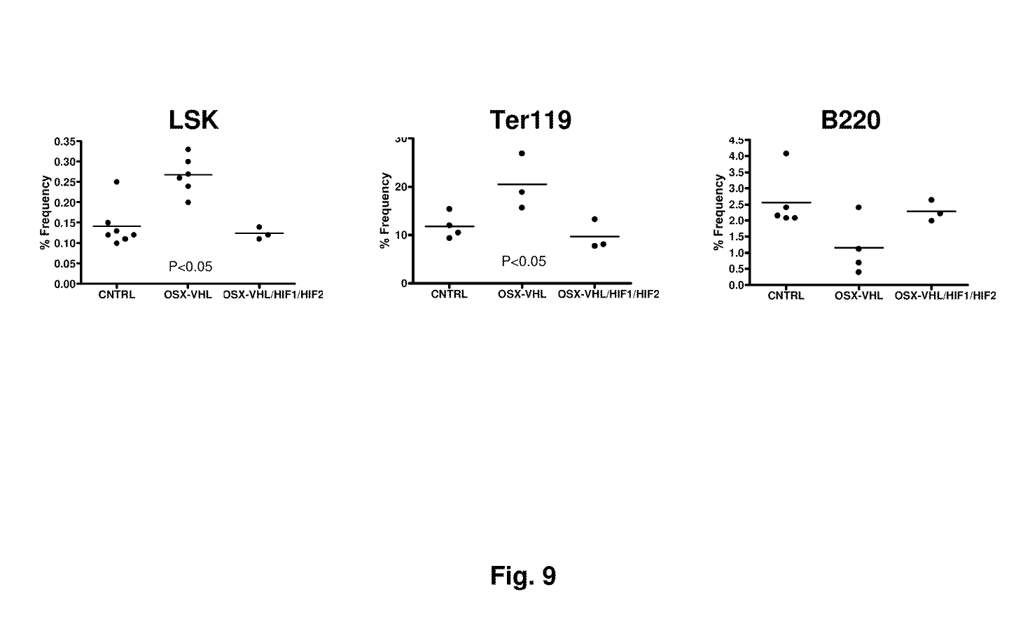

FIG. 10. HIF signaling in osteoprogenitors regulates Epo production.

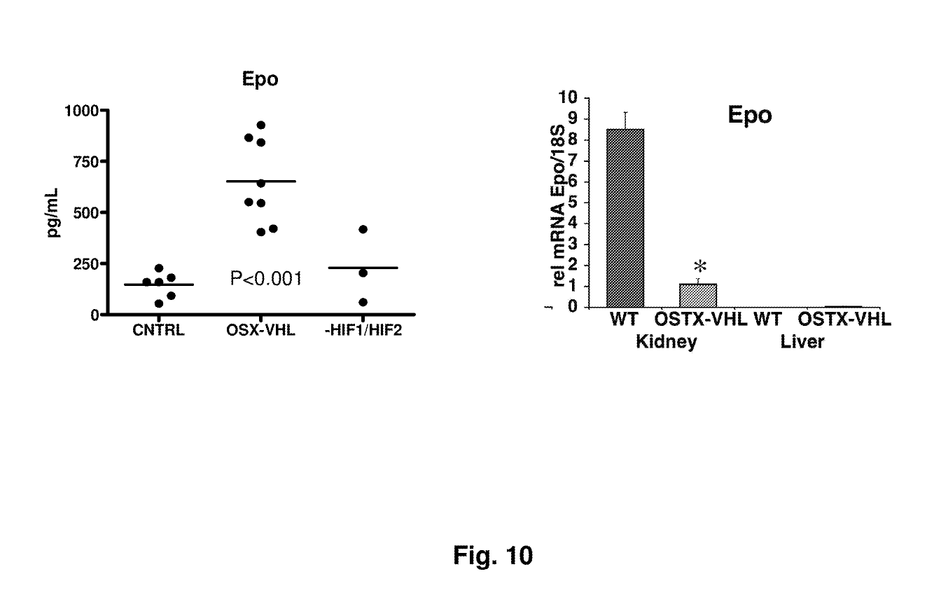

FIG. 11. Schematic of what is happening in hematopoietic development when HIF is constitutively active.

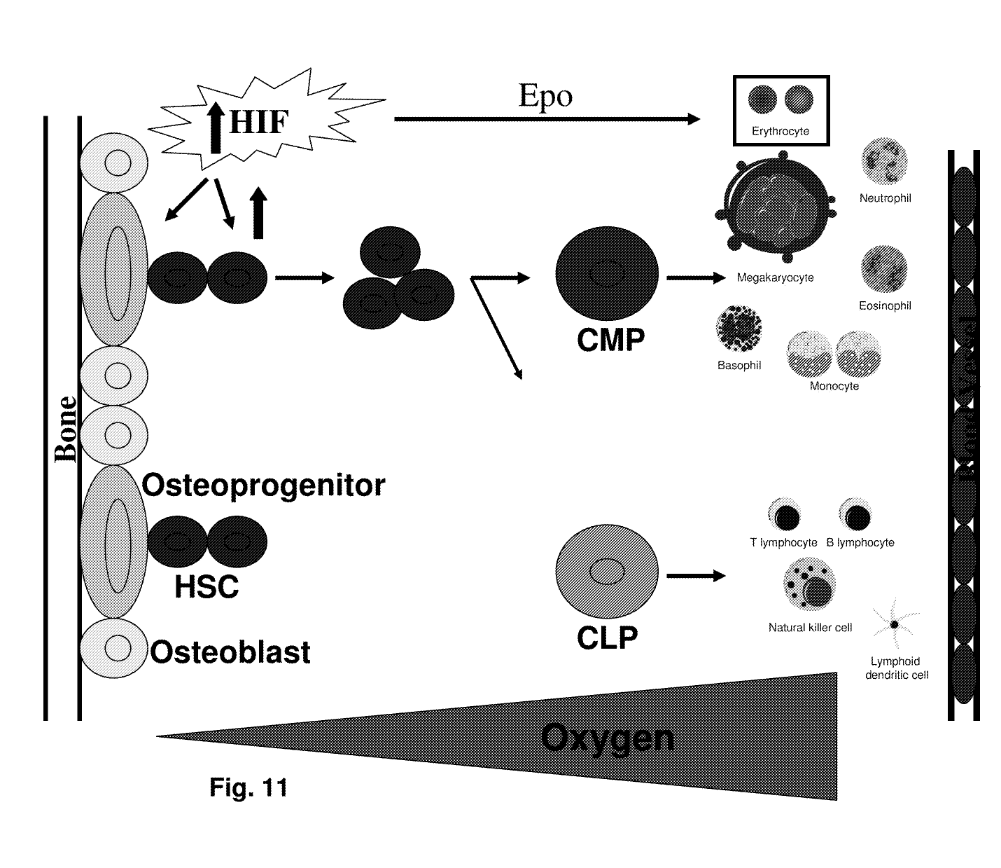

### DETAILED DESCRIPTION OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Methods for promoting the production of erythropoietin and the production of erythrocytes and hematopoietic stem and progenitor cells are provided. These methods find us in the treatment of subjects with conditions in which erythrocyte cell numbers are reduced, for example, anemia, chronic kidney disease, and following chemotherapy treatment. These and other objects, advantages, and features of the invention will become apparent to those persons skilled in the art upon reading the details of the compositions and methods as more fully described below.

By erythropoietin, “EPO”, “hematopoietin” or “hemopoietin” it is meant a secreted, glycosylated cytokine composed of four alpha helical bundles. The protein is found in the plasma and regulates red cell production by promoting erythroid differentiation and initiating hemoglobin synthesis. This protein also has neuroprotective activity against a variety of potential brain injuries and antiapoptotic functions in several tissue types. The amino acid sequence encoding EPO may be found at GenBank Accession No. NM_{—}000799.2.

By “cells of the hematopoietic lineage” it is meant any cells that are derived from hematopoietic stem cells. Cells of the hematopoietic lineage include monocytes; macrophages; neutrophils; basophils; eosinophils; granulocytes; dendritic cells; platelets; erythrocytes (red blood cells); NK cells; osteoclasts; lymphocytes; and progenitor cells thereof.

By a “hematopoietic stem cell” or “HSC” it is meant a cell that can a) self-renew and b) differentiate to produce all mature blood cell types. Hematopoietic stem cells are identifiable in humans by the following combination of markers, without limitation: Lin⁻CD34⁺CD38⁻CD90⁺CD45RA⁻.

By a “hematopoietic progenitor cell” it is meant a descendent of a hematopoietic progenitor that may give rise to a subpopulation of cells of the hematopoietic lineage. For example, the earliest known lymphoid-restricted cell in adult mouse bone marrow is the common lymphocyte progenitor (CLP), and the earliest known myeloid-restricted cell is the common myeloid progenitor (CMP). A complete description of these cell subsets may be found in Akashi et al. (2000) Nature 404(6774):193, U.S. Pat. No. 6,465,247; and published application U.S. Ser. No. 09/956,279 (common myeloid progenitor); Kondo et al. (1997) Cell 91(5):661-7, and International application WO99/10478 (common lymphoid progenitor); and is reviewed by Kondo et al. (2003) Annu Rev Immunol. 21:759-806, each of which is herein specifically incorporated by reference.

By “osteoblast” it is meant a mononucleate cells that is responsible for bone formation. Osteoblasts express many genes expressed by fibroblasts. In addition, they express, without limitation, the osteoblast-specific gene osterix (OSX, also known as SP7), the sequence of which can be found at GenBank Accession Nos. NM_{—}001173467.1 and NM_{—}152860.

By a “hypoxia inducible factor” or “HIF” it is meant a protein whose activity is induced by hypoxic conditions. Examples of HIFs include hypoxia inducible factor 1 alpha (HIF1A), hypoxia inducible factor 2 alpha (HIF2A/EPAS1), and/or hypoxia inducible factor 3 alpha (HIF3A).

By a “hypoxia inducible factor activating agent” or “HIF activating agent” it is meant an agent that promotes the transcriptional activity of a HIF.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

In methods of the present invention, osteoblasts are contacted with a HIF activating agent. As discussed above, a HIF activating agent is an agent that promotes the transcriptional activity of a HIF. Examples of HIF activating agents that promote the transcriptional activity of the HIF include a) those that promote the stability of a HIF (i.e. prevent ubiquitin-dependent degradation of the HIF) e.g. inhibitors of the Von Hippel-Lindau (VHL) protein (e.g. VHL siRNA) and small molecule inhibitors that block the ubiquitination site on HIF; and b) those that promote HIF transcriptional activity on the DNA, e.g. an aryl hydrocarbon receptor nuclear translocator (ARNT) polypeptide or a nucleic acid encoding an ARNT polypeptide; an ARNT2 polypeptide or a nucleic acid encoding an ARNT2 polypeptide; and a CBP/p300 polypeptide or a nucleic acid encoding a CBP/p300 polypeptide.

As suggested by the examples of HIF activating agents above, the HIF activating agent may be a polypeptide, a nucleic acid, or a small molecule compound. In some embodiments, the HIF activating agent that is provided is a polypeptide. To promote transport of HIF activating agents across the cell membrane, HIF activating agent sequences may be fused to a polypeptide permeant domain, e.g. the third alpha helix of Drosophila melanogaster transcription factor Antennapaedia, referred to as penetratin; the HIV-1 tat basic region amino acid sequence, comprising amino acids 49-57 of naturally-occurring tat protein; poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

The HIF activating agent polypeptide may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Other methods of preparing polypeptides in a cell-free system include, for example, those methods taught in U.S. Application Ser. No. 61/271,000, which is incorporated herein by reference.

The HIF activating agent polypeptide may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein. HIF activating agents may be produced recombinantly not only directly, but also as a fusion polypeptide with a heterologous polypeptide, e.g. a polypeptide having a specific cleavage site at the N-terminus of the mature protein or polypeptide. Expression vectors usually contain a selection gene, also termed a selectable marker. This gene encodes a protein necessary for the survival or growth of transformed host cells grown in a selective culture medium.

Following purification by commonly known methods in the art, HIF activating agent polypeptides are provided to the osteoblasts by standard protein transduction methods. In some cases, the protein transduction method includes contacting cells with a composition containing a carrier agent and at least one purified HIF activating agent. Examples of suitable carrier agents and methods for their use include, but are not limited to, commercially available reagents such as Chariot™ (Active Motif, Inc., Carlsbad, Calif.) described in U.S. Pat. No. 6,841,535; Bioport™ (Gene Therapy Systems, Inc., San Diego, Calif.), GenomeONE (Cosmo Bio Co., Ltd., Tokyo, Japan), and ProteoJuice™ (Novagen, Madison, Wis.), or nanoparticle protein transduction reagents as described in, e.g., U.S. patent application Ser. No. 10/138,593.

In other embodiments, the one or more HIF activating agents are nucleic acids. Vectors used for providing nucleic acids to the osteoblasts will typically comprise suitable promoters for driving the expression, that is, transcriptional activation, of the nucleic acids. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by at least about 10-fold, by at least about 100-fold, more usually by at least about 1000-fold. In addition, vectors used for providing the nucleic acids may include genes that must later be removed, e.g. using a recombinase system such as Cre/Lox, or the cells that express them destroyed, e.g. by including genes that allow selective toxicity such as herpesvirus TK, bcl-xs, etc

HIF activating agents that are nucleic acids may be provided directly to the osteoblasts. In other words, the cells are contacted with vectors comprising the nucleic acids such that the vectors are taken up by the cells. Methods for contacting cells with nucleic acid vectors, such as electroporation, calcium chloride transfection, and lipofection, are well known in the art. Vectors that deliver nucleic acids in this manner are usually maintained episomally, e.g. as plasmids or minicircle DNAs.

Alternatively, the nucleic acid may be provided to the osteoblasts via a virus. In other words, the cells are contacted with viral particles comprising the nucleic acids. Retroviruses, for example, lentiviruses, are particularly suitable to such methods. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the osteoblasts are targeted by the packaged viral particles. Methods of introducing the retroviral vectors comprising the nucleic acids into packaging cell lines and of collecting the viral particles that are generated by the packaging lines are well known in the art.

In other embodiments, the HIF activating agent that is provided is a small molecule compound. Small molecule compounds may be provided in any convenient solution that is non-toxic to the osteoblasts, e.g. any culture media, e.g. DMEM, Iscoves, etc. or PBS Typically, the small molecule compound is dissolved in DMSO prior to mixing with the solution that is being used to deliver the small molecule compound to the cells.

When more than one HIF activating agents is provided, the agents may be provided individually or as a single composition, that is, as a premixed composition, of factors. The agents may be added to the osteoblasts simultaneously or sequentially at different times. They may be provided at the same molar ratio or at different molar ratios. The agents may be provided once or multiple times in the course of culturing the osteoblasts. For example, the agent(s) may be provided to the osteoblasts one or more times and the cells allowed to incubate with the agents for some amount of time following each contacting event, e.g. 16-24 hours, after which time the media is replaced with fresh media and the cells are cultured further.

In some embodiments, the osteoblast is contacted with the HIF activating agent in vitro. In such cases, HIF activating agent may be provided in any culture media known in the art to promote cell survival, e.g. DMEM, Iscoves, etc. In some cases, the media will be DMEM. Media may be supplemented with agents that inhibit the growth of bacterial or yeast, e.g. penicillin/streptomycin, a fungicide, etc., with agents that promote health, e.g. glutamate, and other agents typically provided to culture media as are known in the art of tissue culture.

In some embodiments, the osteoblast is contacted with the HIF activating agent in vivo, in which case it may be administered in any physiologically acceptable medium The HIF activating agent may be administered systemically or locally. Any convenient route of administration that results in systemic delivery may be used, e.g. it may be delivered orally, parenterally, intravenously, intracranially, or into spinal fluid. The cells may be introduced by injection, catheter, or the like. The number of administrations of treatment to a subject may vary. Introducing the iNs into the subject may be a one-time event; but in certain situations, such treatment may elicit improvement for a limited period of time and require an on-going series of repeated treatments. In other situations, multiple administrations of the iNs may be required before an effect is observed. The exact protocols depend upon the disease or condition, the stage of the disease and parameters of the individual subject being treated.

In some embodiments, the osteoblast is contacted with the HIF activating agent in vitro, i.e. ex vivo, and the contacted cells are transplanted to a subject for continued growth/survival in vivo. In such cases, the contacted osteoblasts may be administered in any physiologically acceptable medium. They may be provided alone or with a suitable substrate or matrix, e.g. to support their growth and/or organization in the tissue to which they are being transplanted. Usually, at least 1×10⁵ cells will be administered, preferably 1×10⁶ or more. The cells may be introduced to the subject via any of the following routes: parenteral, intravenous, intracranial, intraspinal, intraocular, or into spinal fluid. In some embodiments, they are introduced directly to the bone marrow, e.g. by injection or the like, as is known in the art.

Contacting osteoblasts with HIF activating agents finds use in promoting the cellular production of erythropoietin (EPO) in vitro, e.g. for purification and use in the treatment of disorders or conditions in which the number of circulating erythrocytes is lower than normal, e.g. following chemotherapy or radiation therapy for cancer, anemia, or chronic kidney disease. Contacting osteoblasts with HIF activating agents likewise finds use in promoting the cellular production of erythropoietin (EPO) in vivo for these same purposes. Following contacting osteoblasts with HIF activating agents, the levels of EPO may increase 1.5-fold, 2-fold, 3-fold, 4-fold, 5-fold, 6-fold, 8-fold, or 10-fold, or more. In other words, EPO levels secreted in the medium in vitro or circulating in the blood in vivo may be about 1.5-fold, 2-fold, 3-fold, 4-fold, 5-fold, 6-fold, 8-fold, 10-fold, 15-fold, or 20-fold or more higher following contacting the osteoblasts with HIF activating agents than prior to contacting the osteoblasts. EPO levels may be measured by any convenient method known in the art, e.g. ELISAs, Westerns, or other antibody-based assays.

Contacting osteoblasts with HIF activating agents also finds use in promoting the production of erythrocytes from hematopoietic stem and progenitor cells in vitro, e.g. for transplantation to a patient following chemotherapy or radiation therapy for cancer, or to a patient with a disorder characterized by a lower than normal count of circulating erythrocytes, e.g. anemia or chronic kidney disease, to boost erythrocyte numbers. Likewise, contacting osteoblasts with HIF activating agents also finds use in promoting the production of erythrocytes from hematopoietic stem and progenitor cells in vivo, e.g. in the treatment of these aforementioned diseases and conditions. Increases in erythrocyte numbers may be quantified by any convenient method in the art, e.g. flow cytometry of circulating blood or of bone marrow for the cell marker Ter118, differential cell counts on a blood smear, etc. Erythrocyte numbers will typically rise about 1.5-fold, 2-fold, 3-fold, 4-fold, 5-fold, 6-fold, 8-fold, or 10-fold, or more following contacting the osteoblasts with a HIF activating agent.

Contacting osteoblasts with HIF activating agents also finds use in promoting the production of hematopoietic stem cells, e.g. to increase the population of hematopoietic cells in the body, e.g. for the treatment of hematopoietic conditions and disorders. The increase in hematopoietic stem cells may be quantified by any convenient method in the art, e.g. flow cytometry of a bone marrow sample for the population of cells that is Lin⁻CD34⁺CD38⁻CD90⁺CD45RA⁻. Hematopoietic stem cells numbers will typically rise about 1.5-fold, 2-fold, 3-fold, 4-fold, 5-fold, 6-fold, 8-fold, or 10-fold, or more following contacting the osteoblasts with a HIF activating agent.

### EXAMPLES

The following examples are put forth in the figures and figure legends are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of promoting the cellular production of erythropoietin (EPO) comprising the steps of:
contacting an osteoblast with a hypoxia inducible factor (HIF) activating agent under conditions sufficient to promote the survival of said osteoblast,
wherein EPO is produced.

2. The method of claim 1, wherein said HIF activating agent promotes the transcriptional activity of a hypoxia inducible factor (HIF).

3. The method of 2, wherein said transcriptional activity of a HIF is promoted by stabilizing said HIF.

4. The method of claim 2, wherein said HIF is selected from the group consisting of hypoxia inducible factor 1 alpha (HIF1A), hypoxia inducible factor 2 alpha (HIF2A/EPAS1), and hypoxia inducible factor 3 alpha (HIF3A).

5. The method of claim 1, wherein said contacting step occurs in vitro.

6. The method of claim 1, wherein said contacting step occurs in vivo.

7. The method of claim 6, wherein said HIF activating agent is administered directly to the bone marrow.

8. A method of promoting the production of cells of the hematopoietic lineage from a population of hematopoietic progenitor cells, comprising:
contacting an osteoblast with a HIF activating agent,
culturing a population of hematopoietic progenitor cells in the presence of said osteoblast under conditions sufficient to promote the survival of said hematopoietic progenitor cells,
wherein cells of the hematopoietic lineage are produced.

9. The method of claim 8, wherein said cells of the hematopoietic lineage that are produced comprise erythrocytes.

10. The method of claim 9, wherein the number of erythrocytes that is produced is increased relative to the number of erythrocytes that would be produced in the absence of said HIF activating agent.

11. The method of claim 8, wherein said cells of the hematopoietic lineage that are produced comprise hematopoietic stem and/or progenitor cells.

12. The method of claim 11, wherein the number of hematopoietic stem and/or progenitor cells that is produced is increased relative to the number of hematopoietic stem and/or progenitor cells that would be produced in the absence of said HIF activating agent.

13. The method of claim 8, wherein said contacting step and said culturing step occur in vitro, and said contacting step occurs concurrently with said contacting step.

14. The method of claim 8, wherein said contacting step and said culturing step occur in vitro, and said contacting step occurs prior to said culturing step.

15. The method of claim 8, wherein said contacting step occurs ex vivo and said culturing step occurs in vivo, and said contacting step occurs prior to said culturing step.

16. The method of claim 8, wherein said contacting step and said culturing step occur in vivo, and said contacting step occurs concurrently with said culturing step.

17. The method of claim 16, wherein said HIF activating agent is administered directly to the bone marrow.

18. The method of claim 8, wherein said HIF activating agent promotes the transcriptional activity of a HIF.

19. The method of claim 18, wherein said transcriptional activity is promoted by promoting the stability of said HIF.

20. A method of increasing the number of erythrocytes in a subject in need thereof, comprising:
contacting osteoblasts with a HIF activating agent, and
culturing said osteoblasts in the presence of hematopoietic progenitor cells in said subject,
wherein the number of erythrocytes is increased relative to the number of erythrocytes in the absence of said HIF activating agent.

21. The method of claim 20, wherein erythrocyte production is promoted.

22. The method of claim 20, wherein erythrocytes survival is prolonged.

23. The method of claim 20, wherein the percent of circulating erythrocytes is increased relative to the total number of circulating hematopoietic cells.

24. The method of claim 20, wherein said contacting step results in the production of EPO.

25. The method of claim 20, wherein said contacting step occurs ex vivo prior to said culturing step.

26. The method of claim 20, wherein said contacting step occur in vivo concurrently with said culturing step.

27. The method of claim 8, wherein said HIF activating agent is administered directly to the bone marrow.

28. The method of claim 16, wherein said subject has received or will receive chemotherapy or radiation therapy for cancer, has anemia, or has chronic kidney disease.
